(19) United States
(12) Patent Application Publication
(10) Pub. No.: US 2004/0127873 A1
(43) Pub. Date: Jul. 1, 2004

(54) Absorbent article including porous separation layer with capillary gradient

(76) Inventors: Eugenio Go Varona, Marietta, GA (US); Marlene Rebecca Waugaman, Alpharetta, GA (US); Carol Ann Blaney, Roswell, GA (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/334,653

(22) Filed: Dec. 31, 2002

(51) Int. Cl. A61F 13/15; A61F 13/20

(52) U.S. Cl. 604/385.01

(57) ABSTRACT

An absorbent article including a breathable outer cover, a topsheet, an absorbent core disposed between the topsheet and the breathable outer cover, and a porous separation layer disposed between the absorbent core and the breathable outer cover is disclosed. The porous separation layer includes a plurality of wettable pores originating on a first surface of the porous separation layer adjacent the absorbent core and a plurality of larger wettable pores originating on a second surface adjacent the breathable outer cover.

### FIELD OF INVENTION

The present invention relates to an absorbent article including a porous separation layer disposed between an absorbent core and a breathable outer cover. The porous separation layer includes a plurality of wettable pores originating on a surface adjacent the absorbent core and a plurality of larger, wettable pores originating on a surface adjacent the breathable outer cover.

### BACKGROUND OF THE INVENTION

Many of today's absorbent garments are designed to be highly breathable in order to provide wearer comfort. Such breathable absorbent garments typically utilize breathable outer cover materials that are substantially impermeable to liquids, but are water vapor permeable. The breathable outer cover materials allow escape of water vapor from the absorbent garment, increasing garment comfort and reducing skin rashes and other irritations that result when water vapor is trapped inside the garment and heated by the wearer's body.

Unfortunately, one side effect of providing a breathable outer cover in an absorbent garment is the development of a cold, damp or clammy feel on the outside of the garment. As water in the absorbent core evaporates due to the wearer's body heat and passes through the breathable outer cover material, evaporative cooling occurs. The evaporative cooling causes a decrease in the temperature of the absorbent core and adjacent outer cover material resulting in a clammy, damp-feeling outer cover.

In an effort to overcome this effect, designers of absorbent garments have included a spacer or barrier layer between the absorbent core and the breathable outer cover material. These barrier layers typically include substantially hydrophobic materials such as nonwoven webs that have small, tight-pored structures and/or porous film layers that inhibit moisture from leaving the absorbent core but still allow water vapor to pass through. This results in a gas/vapor layer that thermally insulates the breathable outer cover from the cold and wet absorbent core and reduces the perception of dampness on the outside surface of the absorbent garment.

Absorbent garments may typically include a layer of porous surge material between the absorbent core and the topsheet that assists, through a capillary action mechanism and void volume, in containing the insult volume and pulling fluid into the absorbent core and away from the skin of a wearer thereby resulting in drier, healthier skin. In order to improve the ability of the absorbent core to rapidly take in and distribute fluid, these surge materials are at least partially hydrophilic and have a substantially open structure including large pores that provide the desired capillary action.

These surge materials could be used as a spacer, separation or barrier layer between an absorbent core and an outer cover. Unfortunately, due to the desirable open pore structure of these surge materials, moisture often collects in material. Thus, when these types of surge layers are included between an absorbent core and an outer cover in an absorbent garment having a breathable outer cover, the intake and distribution of fluid into the absorbent core is improved but the evaporative cooling effect noted above can be compounded. On the other, hand utilizing a tight-pored, substantially hydrophobic barrier layer decreases the evaporative cooling effect but does not provide the desired level of capillary action to promote intake and distribution of fluid into the absorbent core.

Therefore there is a need or desire in the absorbent garment industry for an absorbent garment that includes a porous separation layer between an absorbent core and a breathable outer cover material that both: (a) reduces perceived dampness on the outside of the garment; and (b) assists intake and distribution of fluid within the absorbent core.

### SUMMARY OF THE INVENTION

The present invention is directed to an absorbent article having improved fluid intake and reduced perceived outer cover dampness. The absorbent article includes at least a liquid-pervious topsheet, a breathable, substantially liquid-impervious outer cover and an absorbent core between the topsheet and the outer cover. In accordance with the invention, a porous separation layer is disposed between the absorbent core and the breathable outer cover. The porous separation layer includes a plurality of wettable pores originating on a first surface adjacent the absorbent core and a plurality of larger, wettable pores originating on a second surface adjacent the breathable outer cover.

Suitably, the pores originating on the second surface of the porous separation layer have an average pore size proximate the second surface at least about 25 percent greater than the pores originating on the first surface of the porous separation layer. The pores originating on the first surface of the porous separation layer may have an average pore size proximate the first surface of at least about 100 microns and the pores originating on the second surface of porous separation layer may have an average pore size proximate the second surface up to about 1000 microns.

In another embodiment, the pores originating on the first surface of the porous separation layer and the pores originating on the second surface of the porous separation layer may define a pore size gradient from the first surface to the second surface of the porous separation layer. Suitably, the pore size increases by at least about 25 percent from the first surface of the porous separation layer to the second surface of the porous separation layer. In one aspect, the pores originating on the first surface of the porous separation layer and the pores originating on the second surface of the porous separation layer define a pore size gradient that increases from about 100 microns proximate the first surface to about 1000 microns proximate the second surface of the porous separation layer.

In a further embodiment, the pores originating on the second surface of the porous separation layer may be less wettable than the pores originating on the first surface of the porous separation layer. Suitably, the pores originating on the first surface of the porous separation layer may be at least about 5 percent more wettable than the pores originating on the second surface of the porous separation layer, desirably about 10 to about 95 percent more wettable.

In yet another embodiment, the pores originating on the first surface of the porous separation layer and the pores originating on the second surface of the porous separation layer may define a wettability gradient from the first surface to the second surface of the porous separation layer. Suitably, the wettability decreases by at least 5 percent from the first surface to the second surface, desirably about 10 to about 95 percent.

With the foregoing in mind, it is a feature and advantage of the invention to provide an absorbent article including a porous separation layer disposed between an absorbent core and a breathable outer cover that assists in taking in and distributing fluid within the absorbent core and that also decreases perceived dampness on the outside surface of the breathable outer cover.

### BRIEF DESCRIPTION OF THE DRAWINGS

These and other objects and features of this invention will be better understood from the following detailed description taken in conjunction with the drawings, wherein:

FIG. 1 is a plan view of a representative absorbent article of the present invention wherein a portion of the top sheet has been cut away to show the underlying structure of the article.

FIG. 2 is a cross-sectional view taken across Line 2-2 in FIG. 1.

FIGS. 3a and 3b are surface view of a porous separation layer of the present invention.

FIG. 4 is a cross-sectional view of an absorbent article of the invention, illustrating a breathable outer cover.

FIG. 5 is a cross-sectional view of an absorbent article of the invention, illustrating an apertured absorbent core.

### DEFINITIONS

The terms “breathable film” or “breathable outer cover material” refer to a film or outer cover material having a water vapor transmission rate (“WVTR”) of at least about 300 grams/m²-24 hours, using the WVTR Test Procedure described herein.

The term “liquid-permeable material” refers to a material present in one or more layers, such as a film, nonwoven fabric, or open-celled foam, which is porous, and which is water permeable due to the flow of water and other aqueous liquids through the pores. The pores in the film or foam, or spaces between fibers or filaments in a nonwoven web, are large enough and frequent enough to permit leakage and flow of liquid water through the material.

The term “porous material” refers to a material that includes cells or voids that interconnect or otherwise align to form channels or pores from one surface of the material to another surface of the material.

The term “pore size” or “average pore size” refers to the radius of a pore originating on a surface of a porous material as determined according to the Capillary Tension Test described herein.

The term “proximate” or “proximate to” when referring to the average pore size of pores originating on a surface of a porous material refers to the average pore radius measured at a depth within the porous material of less than about 10 microns, suitably less than about 5 microns, and desirably about less than about 1 micron from the surface of the material.

The term “hydrophilic” describes materials that are wetted by an aqueous liquid in contact with a surface of the material. The degree of wetting or “wettability” of the materials can be described in terms of the contact angle between a liquid droplet and a surface across which it spreads. Equipment and techniques suitable for measuring the wettability of particular materials can be provided by a Cahn SFA-222 Surface Force Analyzer System. When measured in accordance with the procedure describe below in detail, materials having contact angles less than 90 degrees are designated wettable, while materials having contact angles greater than 90 degrees are designated “nonwettable”. A change in wettability, such as a decrease in wettability, may be expressed as the percentage change in the cosine of the contact angle, as measured using the Cahn system, from one point to another point such as, for example, the change in the cosine of the contact angle measured at various locations within a pore or channel of a porous separation layer.

The term “nonwoven fabric or web” means a web having a structure of individual fibers or threads which are interlaid, but not in a regular or identifiable manner as in a knitted fabric. Nonwoven fabrics or webs have been formed from many processes such as, for example, meltblowing processes, spunbonding processes, air laying processes, and bonded carded web processes. Pulp or cellulose-based webs are also nonwoven webs. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91.)

The term “poly(lactic acid)” refers to a fiber-forming polymer that is biodegradable and more wettable than polypropylene or polyethylene. Poly(lactic acid) fibers may be used as a total or partial replacement for the synthetic fibers used to form nonwoven fabrics such as those described below.

The term “surge material” refers to a nonwoven fabric or web having an open, porous structure that includes pathways for vapor and/or liquid from one surface to an opposing surface. Examples of such material are disclosed in, for example, U.S. Pat. No. 5,364,382 to Latimer et al., manufactured by Kimberly-Clark Worldwide, Inc.

The term “bonded carded web” refers to webs that are made from staple fibers which are sent through a combing or carding unit, which separates or breaks apart and aligns the staple fibers in the machine direction to form a generally machine direction-oriented fibrous nonwoven web. Such fibers are usually purchased in bales which are placed in an opener/blender or picker which separates the fibers prior to the carding unit. Once the web is formed, it then is bonded by one or more of several known bonding methods. One such bonding method is powder bonding, wherein a powdered adhesive is distributed through the web and then activated, usually by heating the web and adhesive with hot air. Another suitable bonding method is pattern bonding, wherein heated calender rolls or ultrasonic bonding equipment are used to bond the fibers together, usually in a localized bond pattern, though the web can be bonded across its entire surface if so desired. Another suitable and well known bonding method, particularly when using bicomponent staple fibers, is through-air bonding.

The term “microfibers” means small diameter fibers typically having an average fiber denier of about 0.005 to 10. Fiber denier is defined as grams per 9000 meters of fiber. For a fiber having a circular cross-section, denier may be calculated as fiber diameter in microns squared, multiplied by the density in grams per cubic centimeter (g/cc) multiplied by 0.00707. For fibers made of the same polymer, a lower denier indicates a finer fiber and a higher denier indicates a thicker or heavier fiber. For example, the diameter of a polypropylene fiber given as 15 microns may be converted to denier by squaring, multiplying the result by 0.89 g/cc and multiplying that result by 0.00707. Thus, a 15 micron polypropylene has a denier of about 1.42 calculated as (15²×0.89×0.00707=1.415). Outside the United States the unit of measurement is more commonly the “tex” which is defined as grams per kilometer of fiber. Tex may be calculated as denier/9.

The term “spunbond fibers” refers to small diameter fibers which are formed by extruding molten thermoplastic material as filaments from a plurality of fine capillaries of a spinneret having a circular or other configuration, with the diameter of the extruded filaments then being rapidly reduced as in, for example, U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein in its entirety by reference. Spunbond fibers are quenched and generally not tacky when they are deposited onto a collecting surface. Spunbond fibers are generally continuous and often have average deniers larger than about 0.3, more particularly, between about 0.6 and 10.

The term “meltblown fibers” means fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers that may be continuous, are generally smaller than about 1.0 denier, and are generally self-bonding when deposited onto a collecting surface.

The term “interfiber bonding” means bonding produced by thermal bonding or entanglement between the individual nonwoven fibers to form a coherent web structure. Fiber entangling is inherent in the meltblown process but may be generated or increased by processes such as, for example, hydraulic entangling or needlepunching. One or more thermal bonding steps are employed in most processes for forming spunbond webs. Alternatively and/or additionally, a bonding agent can be utilized to increase the desired bonding and to maintain structural coherency of the web. For example, powdered bonding agents and chemical solvent bonding may be used.

The term “film” refers to a thermoplastic film made using a film extrusion process, such as a cast film or blown film extrusion process. This term includes films rendered microporous by mixing a polymer with filler, forming a film from the mixture, and stretching the film.

The term “polymer” includes, but is not limited to, homopolymers, copolymers, such as for example, block, graft, random and alternating copolymers, terpolymers, etc. and blends, and modifications thereof. Additionally, the term “polymer” includes thermoplastic and thermoset polymers. Furthermore, unless otherwise specifically limited, the term “polymer” shall include all possible geometrical configurations of the material. These configurations include, but are not limited to, isotactic, syndiotactic and atactic symmetries.

The term “foam material” refers to a thermoplastic layer material made with the aid of a foaming process. The term “open-celled foam material” refers to a foam layer that includes cells that interconnect, or otherwise create pores from one surface of the layer to the opposite surface.

The term “superabsorbent” or “superabsorbent material” refers to a water-swellable, water-insoluble organic or inorganic material capable, under the most favorable conditions, of absorbing at least about 20 times its weight and, more desirably, at least about 30 times its weight in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds such as cross-linked polymers. The term “cross-linked” refers to any means for effectively rendering normally water-soluble materials substantially water insoluble but swellable. Such means can include, for example, physical entanglement, crystalline domains, covalent bonds, ionic complexes and associations, hydrophilic associations, such as hydrogen bonding, and Van der Waals forces.

The term “garment” includes pant-like absorbent garments and medical and industrial protective garments. The term “pant-like absorbent garment” includes without limitation diapers, training pants, swim wear, absorbent underpants, baby wipes, adult incontinence products, and feminine hygiene products.

The term “medical protective garment” includes without limitation surgical garments, gowns, aprons, facemasks, and drapes. The term “industrial protective garment” includes without limitation protective uniforms and workwear.

### DESCRIPTION OF PREFERRED EMBODIMENTS

The present invention relates to absorbent articles including a porous separation layer disposed between an absorbent core and a breathable outer cover. For ease of explanation, the description hereafter will be in terms of a diaper. However, it should be understood that other absorbent articles such as, but not limited to, child training pants, adult incontinence garments, swim wear, feminine hygiene products and the like are also contemplated.

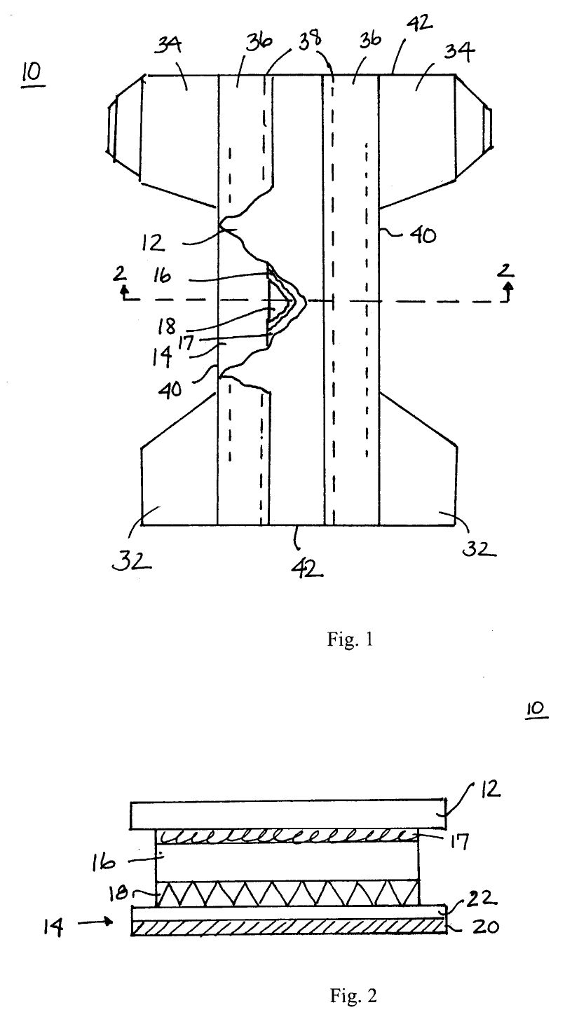

Referring to FIGS. 1 and 2, a diaper 10 includes a liquid-pervious topsheet 12 that is configured to contact the wearer, and a breathable, liquid-impervious outer cover 14 opposite the topsheet that is configured to contact the wearer's clothing. An absorbent core 16 is positioned or disposed between the breathable outer cover 14 and the topsheet 12. A porous separation layer 18 is disposed between the absorbent core 16 and the breathable outer cover 14.

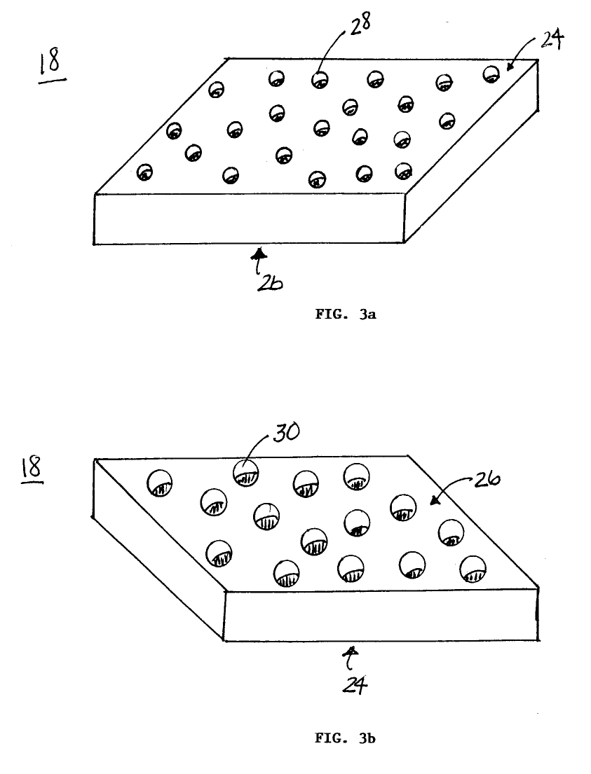

Referring to FIGS. 3a and 3b, the porous separation layer includes a first surface 24 and a second surface 26. Advantageously, the first surface 24 is positioned adjacent the absorbent core 16 and the second surface 26 is positioned adjacent the breathable outer cover 14. The first surface 24 of the porous separation layer 18 includes a plurality of wettable pores 28 that originate on the first surface 24 and extend into the bulk of the porous separation layer 18 as shown in FIG. 3a. The second surface 26 of the porous separation layer 18 includes a plurality of larger wettable pores 30 that originate on the second surface 26 and extend into the bulk of the porous separation layer 18 as shown in FIG. 3b. Suitably the pores 28 originating on the first surface 24 and the pores 30 originating on the second surface 26 define a plurality of pathways or channels extending into the porous separation layer 18 that allow vapor to pass from the first surface 24 to the second surface 26 of the porous separation layer. Desirably, the wettable pores 30 originating on the second surface 26, while being available for surge function when needed, release their fluid toward the first surface 24 as fluid is absorbed into the absorbent core due to a capillary gradient. The capillary gradient provides a driving force for excess liquid to be transported toward the absorbent core 16.

Without wishing to be bound by theory, it is believed that configuring the porous separation layer in the above manner maximizes the intake and distribution properties of the absorbent core 16 as well as the insulative properties of the porous separation layer. By placing the first surface 24, having wettable pores 28, adjacent the absorbent core 16 and the second surface 26, having larger wettable pores 30, adjacent the breathable outer cover 14 it is theorized that a capillary gradient is formed. Specifically, as the diaper 10 is insulted with a liquid such as urine, the porous separation layer 18 acts through a capillary mechanism to draw fluid into the absorbent core 16 toward the wettable pores 28 on the first surface 24 of the porous separation layer. Due to the capillary action of the wettable pores originating on the first surface of the porous separation layer the fluid can be drawn into the absorbent core 16 and distributed more quickly. Furthermore, the larger wettable pores 30 encourage capillary desorption of fluid that has entered the porous separation layer 18 back into the absorbent core 16, thereby keeping the porous separation layer relatively dry. As discussed above, reducing the amount of moisture in the barrier layer minimizes the perception of the evaporative cooling effect that contributes to perceived dampness on the outside surface of the breathable outer cover because the insulative properties of the porous separation layer are maintained.

Suitably, the wettable pores 28 originating on the first surface 24 of the porous separation layer have an average pore size proximate the first surface of at least about 100 microns. Advantageously, the wettable pores 30 originating on the second surface 26 of the porous separation layer 18 have an average pore size proximate the second surface that is greater than the average pore size of the wettable pores 28 proximate the first surface 24 of the porous separation layer 18, suitably at least about 25 percent greater, desirably about 30 to about 1000 percent greater, and in one embodiment about 100 to about 300 percent greater. In one aspect, the wettable pores 30 may have an average pore size proximate the second surface up to about 1000 microns. The average pore size of the pores originating on the first and second surfaces of the porous separation material is measured as the average pore radius and may be measured using the Capillary Tension Test described herein.

As shown FIGS. 3a and 3b, the wettable pores 28 appear to be of an approximately equivalent size. Similarly, the wettable pores 30 also appear to be of an approximately equivalent size. It should be understood, that the pore size of the individual pores originating on either the first surface 24 or the second surface 26 may vary from the average pore size dimensions indicated above so long as the average pore size is within in the desired range. Furthermore, the individual pores originating on either the first surface 24 or the second surface 26 of the porous separation layer 18 may be uniformly or non-uniformly positioned on the surfaces.

Advantageously, the wettable pores 28 originating on the first surface 24 of the porous separation layer 18 and the wettable pores 30 originating on the second surface 26 of the porous separation layer 18 define a pore size gradient between the first surface and the second surface of the porous separation layer. Suitably, the average pore size increases by at least about 25 percent from the first surface 24 to the second surface 26 of the porous separation layer. Desirably, the average pore size increase is about 30 to about 1000 percent, suitably about 100 to about 300 percent. In one aspect, the average pore size may range from an average pore size of about 100 microns proximate the first surface 24 to an average pore size of about 1000 microns proximate the second surface 26 of the porous separation layer 18. The pore size gradient between the first and second surfaces of the porous separation layer may be measured using the Capillary Tension Test described herein.

In one embodiment, the larger pores 30 originating on the second surface 26 of the porous separation layer 18 may be less wettable than the wettable pores 28 originating on the first surface 24 of the porous separation layer 18. Without wishing to be bound by theory, it is believed that by configuring the porous separation layer in this manner that the less wettable pores 30 originating on the second surface 26 of the porous separation layer 18 inhibit fluid that enters the porous separation layer from advancing through the layer toward the breathable outer cover 14, thus keeping the porous separation layer relatively dry. Desirably, the wettable pores 30 originating on the second surface 26, while being available for surge function when needed, release their fluid toward the first surface 24 as fluid is absorbed into the absorbent core 16 due to the pore size and/or wettability gradient. The pore size and/or wettability gradient provides a driving force for excess liquid to be transported toward the absorbent core 16. Wettability and change in wettability may be determined using the Cahn Test described herein.

Desirably, the larger wettable pores 30 originating on the second surface 26 of the porous separation layer 18 are at least about 5 percent less wettable than the wettable pores 28 originating on the first surface 24 of the porous separation layer 18, suitably about 10 to about 95 percent less wettable, and in one aspect, about 50 to about 85 percent less wettable.

Advantageously, the wettable pores 28 originating on the first surface 24 of the porous separation layer 18 and the wettable pores 30 originating on the second surface 26 of the porous separation layer 18 define a wettability gradient between the first surface and the second surface of the porous separation layer. Suitably, the wettability decreases by at least about 5 percent from the first surface 24 to the second surface 26 of the porous separation layer. Desirably, the percentage decrease in wettability is about 10 to about 95 percent, preferably about 50 to about 85 percent.

The various layers of the diaper 10 have dimensions that vary depending on the size and shape of the wearer. As shown in FIGS. 1 and 2, the topsheet 12 and the breathable outer cover 14 are generally coextensive and have a width dimension generally larger than that of the absorbent core 16 and the porous separation layer 18. Furthermore, the absorbent core 16 and the porous separation layer 18 are shown as having the generally same width dimensions. It should be understood, however, that the various layers of the diaper 10 may be assembled in a variety of configurations to provide the desired level of functionality. For example, the porous separation layer 18 may be configured to include length and/or width dimensions that are larger or smaller than those of the absorbent core. In another aspect, the porous separation layer 18 may be configured to be of any desired shape consistent with the absorbent and insulative requirements of the diaper 10. Suitable shapes include, for example circular, rectangular, triangular, trapezoidal, oblong, dog-boned, hourglass-shaped, or oval. Generally, those shapes that increase the liquid-communicating surface area between the absorbent core 16 and the porous separation layer 18, so that the relative capillarity differences between the two layers can be fully utilized, are desired.

Suitably, the porous separation layer 18 may have a basis weight of at least about 20 grams per square meter (gsm), desirably within the range of about 20 gsm to about 120 gsm. The porous separation layer 18 may be constructed from porous woven materials, porous nonwoven materials, and apertured films. Examples include, without limitation, any flexible porous sheets of polyolefin fibers, such as polypropylene, polyethylene or polyester fibers; webs of spunbonded polypropylene, polyethylene or polyester fibers; webs of rayon fibers; bonded carded webs of synthetic or natural fibers or combinations thereof. The porous separation layer may also include one or more layers an apertured plastic film. In one aspect, the porous separation layer 18 may include, in whole or in part, poly(lactic acid) fibers. As noted above, it has been found that poly(lactic acid) fibers have superior wettability as compared to synthetic fibers such as polyethylene and polypropylene as well as the added characteristic of biodegradability which may be desirable in a disposable absorbent article such as are contemplated by the present invention.

In one embodiment, the porous separation layer 18 may include a nonwoven material such as disclosed in U.S. Pat. No. 5,679,042 to Varona, which is incorporated by reference. Suitably, the barrier layer 18 has a generally uniform thickness and cross-sectional area and is at least partially wettable.

Referring again to FIGS. 1 and 2, the liquid-permeable topsheet 12 is illustrated as overlying the breathable outer cover 14 and absorbent core 16, and may but need not have the same dimensions as the breathable outer cover 14. The topsheet 12 is desirably compliant, soft feeling, and non-irritating to the wearer's skin. Further, the topsheet 12 can be less hydrophilic than the absorbent core 16, to present a relatively dry surface to the wearer and permit liquid to readily penetrate through its thickness.

The topsheet 12 can be manufactured from a wide selection of web materials, such as synthetic fibers (for example, polyester or polypropylene fibers), natural fibers (for example, wood or cotton fibers), a combination of natural and synthetic fibers, porous foams, reticulated foams, apertured plastic films, or the like. In one aspect, the topsheet 12 may include, in whole or in part, poly(lactic acid) fibers. It has been found that such poly(lactic acid) fibers are more wettable than many synthetic fibers such as polyethylene and polypropylene. Furthermore, poly(lactic acid) fibers are generally biodegradable. Thus, the use of such fibers within the absorbent articles of the present invention is desirable to improve the disposability aspects of these articles.

Various woven and nonwoven fabrics can be used for the topsheet 12. For example, the topsheet can be composed of a meltblown or spunbonded web of polyolefin fibers. The topsheet can also be a bonded-carded web composed of natural and/or synthetic fibers.

The topsheet can be composed of a substantially hydrophobic material, and the hydrophobic material can, optionally, be treated with a surfactant or otherwise processed to impart a desired level of wettability and hydrophilicity. For example, the material can be surface treated with about 0.45 weight percent of a surfactant mixture including AHCOVEL N-62 available from Uniqema Inc., a division of ICI of New Castle, Del. and GLUCOPON 220UP available from Cognis Corporation of Ambler, Pa., and produced in Cincinnati, Ohio, in an active ratio of 3:1. The surfactant can be applied by any conventional means, such as spraying, printing, brush coating or the like. The surfactant can be applied to the entire topsheet 12 or can be selectively applied to particular sections of the topsheet, such as the medial section along a longitudinal centerline.

The outer cover 14 is breathable to water vapor. Generally the outer cover 14 will have a WVTR of at least about 300 grams/m²-24 hours using the test procedure described below, preferably at least about 1500 grams/m²-24 hours, more preferably at least about 3000 grams/m²-24 hours.

The breathable outer cover 14 desirably includes a multi-layered laminate structure in which at least one of the layers is liquid impermeable. For instance, the breathable outer cover 14 can include a liquid permeable outer layer 20 and a liquid impermeable inner layer 22, as shown in FIG. 2, that are suitably joined together by a laminate adhesive (not shown). Suitable laminate adhesives, which can be applied continuously or intermittently as beads, a spray, parallel swirls, or the like, can be obtained from Findley Adhesives, Inc., of Wauwatosa, Wis., or from National Starch and Chemical Company, Bridgewater, N.J.

The liquid permeable outer layer 20 can be any suitable material and desirably one that provides a generally cloth-like texture. One example of such a material is a 20-gram per square meter (gsm) spunbond polypropylene nonwoven web. The outer layer may also be made of those materials of which the liquid permeable topsheet 12 is made. In another aspect, the liquid-permeable outer layer 20 of the breathable outer cover 14 can be nonwoven web including in whole or in part poly(lactic acid) fibers. The use of such poly(lactic acid) fibers is desirable to impart biodegradability to disposable absorbent article such as are contemplated by the present invention. While it is not a necessity for the outer layer to be liquid permeable, it is desired that it provides a relatively cloth-like texture to the wearer.

The inner layer 22 of the breathable outer cover 14 should be liquid impermeable and vapor permeable. The inner layer 22 is desirably manufactured from a thin plastic film, although other flexible liquid impermeable materials may also be used. The inner layer 22 prevents waste material from wetting articles, such as bedsheets and clothing, as well as the wearer and caregiver.

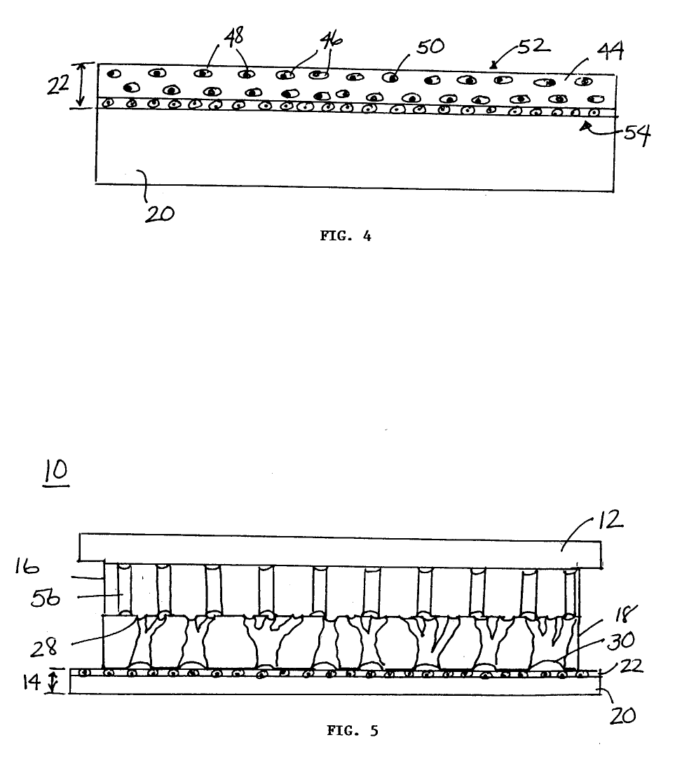

Referring to FIG. 4, the inner layer 22 of the breathable outer cover 14 desirably includes at least one layer of a polymer matrix 44 including a plurality of voids 46 within the matrix surrounded by relatively thin microporous membranes 48 defining tortuous paths, and one or more filler particles 50 in each void 46. The inner layer 22 is microporous and breathable, wherein the microporous membranes between the voids readily permit molecular diffusion of water vapor from a first surface 52 to a second surface 54 of the inner layer 22. A suitable breathable material is composed of a microporous polymer film or a nonwoven fabric that has been coated or otherwise treated to impart a desired level of liquid impermeability.

The polymer matrix 44 can be formed from any suitable film-forming thermoplastic polymer. Examples of suitable polymers include without limitation polyethylene, polypropylene, copolymers of mainly ethylene and C₃-C₁₂ alpha-olefins (commonly known as linear low density polyethylene), copolymers of mainly propylene with ethylene and/or C₄-C₁₂ alpha-olefins, and flexible polyolefins including propylene-based polymers having both atactic and isotactic propylene groups in the main polypropylene chain. Other suitable matrix polymers include without limitation elastomers, for example polyurethanes, copolyether esters, polyamide polyether block copolymers, ethylene vinyl acetate copolymers, block copolymers having the general formula A-B-A′ or A-B such as copoly (styrene/ethylene-butylene), styrene-poly (ethylene-propylene)-styrene, styrene-poly (ethylene-butylene)-styrene, polystyrene/poly(ethylene-butylene)/polystyrene, poly (styrene/ethylene-butylene/styrene), and the like. Metallocene-catalyzed polyolefins are also useful, including those described in U.S. Pat. Nos. 5,571,619; 5,322,728; and 5,272,236, the disclosures of which are incorporated herein by reference.

Polymers made using metallocene catalysts have a very narrow molecular weight range. Polydispersity numbers (Mw/Mn) of below 4 and even below 2 are possible for metallocene-produced polymers. These polymers also have a controlled short chain branching distribution compared to otherwise similar Ziegler-Natta produced type polymers. It is also possible using a metallocene catalyst system to control the isotacticity of the polymer quite closely.

The filler particles 50 can include any suitable inorganic or organic filler. The filler particles 50 are preferably small, in order to maximize vapor transmission through the voids. Generally, the filler particles should have a mean particle diameter of about 0.1-7.0 microns, preferably about 0.5-7.0 microns, most preferably about 0.8-2.0 microns. Suitable fillers include without limitation calcium carbonate, non-swellable clays, silica, alumina, barium sulfate, sodium carbonate, talc, magnesium sulfate, titanium dioxide, zeolites, aluminum sulfate, diatomaceous earth, magnesium sulfate, magnesium carbonate, barium carbonate, kaolin, mica, carbon, calcium oxide, magnesium oxide, aluminum hydroxide and polymer particles. Calcium carbonate is one preferred filler. Linear low density polyethylene (Ziegler-Natta or metallocene-catalyzed, or a blend thereof) is a presently preferred polymer matrix material.

The filler particles may be coated with a minor quantity (e.g. up to 2% by weight) of a fatty acid or other material to ease their dispersion in the polymer matrix. Suitable fatty acids include without limitation stearic acid, or a larger chain fatty acid such as behenic acid. The amount of filler particles in the inner layer 22 should range from about 30-80% by weight of the inner layer 22, preferably about 40-70% by weight, most preferably about 50-65% by weight. Similarly, the polymer matrix 44 should constitute about 20-70% by weight of the inner layer 22, preferably about 30-60% by weight, more preferably about 35-50% by weight.

The polymer composition, filler content, filler particle size and degree of stretching are factors that help determine the breathability of the inner layer 22. Generally, the inner layer 22 will be less than about 50 microns thick, preferably less than about 30 microns thick, most preferably less than about 20 microns thick. The breathable film used to form the inner layer 22 may be uniaxially stretched to about 1.1-7.0 times its original length, preferably to about 1.5-6.0 times its original length, most preferably to about 2.5-5.0 times its original length. The film may alternatively be biaxially stretched using conventional techniques familiar to persons skilled in the art.

One suitable microporous film is a PMP-1 film material commercially available from Mitsui Toatsu Chemicals, Inc., Tokyo, Japan, or an XKO-8044 polyolefin film commercially available from 3M Company, Minneapolis, Minn. Other materials suitable for making the inner layer 22 of the breathable outer cover 14 include monolithic breathable films, such as those made of polyether amide based polymers, for example PEBAX from ATOFINA Chemicals, Inc. of Philadelphia, Pa., and ether/ester polyurethane thermal-plastic elastomers.

Absorbent core 16 can be made of wood pulp fluff or a mixture of wood pulp fluff and a superabsorbent material, or a wood pulp fluff integrated with a thermoplastic absorbent material treated with a surfactant. Thermal binders can be used in blends or layering with the fluff and superabsorbent. The absorbent core 16 can also be a batt of meltblown synthetic fibers, a bonded carded web of synthetic or natural fibers or blends thereof, a composite of meltblown fibers and the like. The synthetic fibers can be, but are not limited to, polypropylene, polyethylene, polyester and copolymers of these or other polyolefins. In another aspect, poly(lactic acid) fibers may be included in the materials of the absorbent 16 in order to alter both the wettability and the biodegradability of the absorbent core 16.

Examples of synthetic superabsorbent material polymers include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinylmorpholinone), poly(vinyl alcohol), and mixtures and copolymers thereof. Further superabsorbent materials include natural and modified natural polymers, such as hydrolyzed acrylonitrile-grafted starch, acrylic acid grafted starch, methyl cellulose, chitosan, carboxymethyl cellulose, hydroxypropyl cellulose, and the natural gums, such as alginates, xanthan gum, locust bean gum and the like. Mixtures of natural and wholly or partially synthetic superabsorbent polymers can also be useful in the present invention. Other suitable absorbent gelling materials are disclosed by Assarsson et al. in U.S. Pat. No. 3,901,236. Processes for preparing synthetic absorbent gelling polymers are disclosed in U.S. Pat. No. 4,076,663 to Masuda et al. and U.S. Pat. No. 4,286,082 to Tsubakimoto et al.

In an alternate embodiment shown in FIG. 5, the absorbent core 16 may be apertured, which greatly increases the number of pathways for moisture to transport itself away from the skin surface. In the embodiment of FIG. 5, the apertures 56 extend through absorbent core 16 and locally correspond to at least a portion of the wettable pores 28 originating on the first surface 24 of the porous separation layer 18.

Referring again to FIGS. 1 and 2, the diaper 10 may optionally include a surge layer 17 disposed between the topsheet 12 and the absorbent core 16 to assist in the collection and distribution of liquid insults within the diaper. Various woven and nonwoven fabrics can be used to construct the surge layer 17. For example, the surge layer 17 may be a layer composed of a meltblown or spunbonded web of synthetic fibers, such as polyolefin fibers. The surge layer 17 may also be a bonded-carded-web or an airlaid web composed of natural and synthetic fibers. The bonded-carded-web may, for example, be a thermally bonded web that is bonded using low melt binder fibers, powder or adhesive. The webs can optionally include a mixture of different fibers. The surge layer 17 may be composed of a substantially hydrophobic material, and the hydrophobic material may optionally be treated with a surfactant or otherwise processed to impart a desired level of wettability and hydrophilicity. In a particular embodiment, the surge layer 17 may include a hydrophobic, nonwoven material having a basis weight of from about 30 to about 120 grams per square meter. One suitable surge material is disclosed in, for example, U.S. Pat. No. 5,364,382 to Latimer et al., which is hereby incorporated by reference.

The diaper 10, as shown in FIG. 1, may also include a number of non-absorbent structural components. For example, the diaper 10 may include a pair of transversely opposed front side panels 32, and a pair of transversely opposed back side panels 34. The side panels 32, 34 may be integrally formed with the breathable outer cover 14 and/or the topsheet 12, or may include two or more separate elements.

Other non-absorbent structural components in the diaper 10 may include a pair of containment flaps 36 which are configured to provide a barrier to the transverse flow of any body exudates discharged from the wearer. A flap elastic member 38 may be operatively joined with each containment flap 36 in any suitable manner as is well known in the art. The elasticized containment flaps 36 define an unattached edge that assumes an upright, generally perpendicular configuration in at least a crotch region of the diaper 10 to form a seal against the wearer's body. The containment flaps 36 can be located along transversely opposed side edges 40 of the diaper 10, and can extend longitudinally along the entire length of the diaper or may only extend partially along the length of the diaper. Suitable constructions and arrangements for the containment flaps 36 are generally well known to those skilled in the art.

Additional non-absorbent structural components (not shown) may include for example, waist elastic positioned adjacent longitudinal ends 42 of the diaper 10, leg elastics (not shown) positioned adjacent the transverse edges 40 of the diaper 10, and various additional layers (not shown) disposed between the topsheet 14 and the breathable outer cover 16 to assist in the collection, retention and distribution of fluids and/other body exudates.

### CAPILLARY TENSION TEST FOR DETEMINING PORE SIZE

The average pore size, measured as average pore radius, and the pore size gradient of a material may be determined by using an apparatus based on the porous plate method first reported in Burgeni and Kapur in the Textile Research Journal, Volume 37, pp. 356-366 (1967). The apparatus is a modified version of the porous plate method and includes a movable Velmex stage interfaced with a programmable stepper motor and an electronic balance controlled by a computer. A control program automatically moves the stage to the desired height, collect data at a specified sampling rate until equilibrium is reached, and then moves to the next calculated height. Controllable parameters of the method include sampling rates, criteria for equilibrium and the number of absorption/desorption cycles.

Data for this analysis are collected using mineral oil in desorption mode. That is, the material is saturated at zero height and the porous plate (and the effective capillary tension on the sample) is progressively raised in discrete steps corresponding to the desired capillary radius. The amount of liquid pulled out from the sample is monitored. Readings at each are taken every 15 seconds and equilibrium is assumed to be reached when the average change of four consecutive readings is less than 0.005 grams. This method is described in more detail in U.S. Pat. No. 5,679,042 to Varona which is hereby incorporated by reference.

### MATERIAL WETTABILITY DETERMINATIONS (CAHN TEST)

The wettability of materials can be determined using contact angle measurements. Repeat cycle, single material contact angle measurements using distilled water are performed with a Cahn Surface Force Analyzer (SFA222) and WET-TEK.RTM data analysis software. The SFA222 is available from Cahn Instruments, Inc. of Cerritos, Calif. and the WET-TEK software is available from Biomaterials International, Inc., of Salt Lake City, Utah. Materials are tested through three measurement cycles, and the distilled water bath is changed between cycles one and two. Materials are determined to be wettable if all three repeat cycles measure a contact angle of less than 90 degrees. Otherwise the materials are deemed “nonwettable”. The test instrument is operated in accordance with the standard operating techniques described in the Cahn SFA-222 System Instruction Manual supplied by the manufacturer.

The change in wettability from one point or surface to another point or surface may be calculated as the percentage difference in the cosine of the contact angle measured at two points on or within a nonwoven material.

### WATER VAPOR TRANSMISSION RATE TEST

A suitable technique for determining the WVTR (water vapor transmission rate) value of a film or laminate material of the invention is the test procedure standardized by INDA (Association of the Nonwoven Fabrics Industry), number IST-70.4-99, entitled “STANDARD TEST METHOD FOR WATER VAPOR TRANSMISSION RATE THROUGH NONWOVEN AND PLASTIC FILM USING A GUARD FILM AND VAPOR PRESSURE SENSOR” which is incorporated by reference herein. The INDA procedure provides for the determination of WVTR, the permeance of the film to water vapor and, for homogeneous materials, water vapor permeability coefficient.

The INDA test method is well known and will not be set forth in detail herein. However, the test procedure is summarized as follows. A dry chamber is separated from a wet chamber of known temperature and humidity by a permanent guard film and the sample material to be tested. The purpose of the guard film is to define a definite air gap and to quiet or still the air in the air gap while the air gap is characterized. The dry chamber, guard film, and the wet chamber make up a diffusion cell in which the test film is sealed. The sample holder is known as the Permatran-W Model 100K manufactured by Mocon/Modem Controls, Inc., Minneapolis, Minn. A first test is made of the WVTR of the guard film and the air gap between an evaporator assembly that generates 100% relative humidity. Water vapor diffuses through the air gap and the guard film and then mixes with a dry gas flow that is proportional to water vapor concentration. The electrical signal is routed to a computer for processing. The computer calculates the transmission rate of the air gap and the guard film and stores the value for further use.

The transmission rate of the guard film and air gap is stored in the computer as CalC. The sample material is then sealed in the test cell. Again, water vapor diffuses through the air gap to the guard film and the test material and then mixes with a dry gas flow that sweeps the test material. Also, again, this mixture is carried to the vapor sensor. The computer than calculates the transmission rate of the combination of the air gap, the guard film, and the test material. This information is then used to calculate the transmission rate at which moisture is transmitted through the test material according to the equation:

TR⁻¹_{test material}=TR⁻¹_{test material, guardfilm, airgap}−TR⁻¹_{guardfilm, airgap}

Calculations:

WVTR: The calculation of the WVTR uses the formula:

WVTR=Fpₛₐₜ(T)RH/Apₛₐₜ(T)(1−RH))

where:

F=The flow of water vapor in cc/min.,

pₛₐₜ(T)=The density of water in saturated air at temperature T,

RH=The relative humidity at specified locations in the cell,

A=The cross sectional area of the cell, and,

pₛₐₜ(T)=The saturation vapor pressure of water vapor at temperature T.

While in the foregoing specification this invention has been described in relation to certain preferred embodiments thereof, and many details have been set forth for purpose of illustration, it will be apparent to those skilled in the art that the invention is susceptible to additional embodiments and that certain of the details described herein can be varied considerably without departing from the basic principles of the invention.

What is claimed is:

1. An absorbent article comprising:
a breathable liquid-impervious outer cover;
a liquid-pervious topsheet;
an absorbent core disposed between the topsheet and the breathable outer cover; and
a porous separation layer disposed between the absorbent core and the breathable outer cover,
wherein the porous separation layer includes a plurality of wettable pores originating on a first surface adjacent the absorbent core and a plurality of wettable pores originating on a second surface adjacent the breathable outer cover, the pores originating on the second surface having a larger average pore size proximate the second surface than the pores originating the first surface.

2. The absorbent article of claim 1, wherein the pores originating on the second surface of the porous separation layer have an average pore size proximate the second surface at least about 25 percent greater than the pores originating on the first surface of the porous separation layer.

3. The absorbent article of claim 1, wherein the pores originating on the second surface of the porous separation layer have an average pore size proximate the second surface about 30 to about 1000 percent greater than the pores originating on the first surface of the porous separation layer.

4. The absorbent article of claim 1, wherein the pores originating on the first surface of the porous separation layer have an average pores size proximate the first surface of at least about 100 microns.

5. The absorbent article of claim 1, wherein the pores originating on the second surface of the porous separation layer have an average pore size proximate the second surface up to about 1000 microns.

6. The absorbent article of claim 1, wherein the pores originating on the first surface of the porous separation layer and the pores originating on the second surface of the porous separation layer define an increasing pore size gradient from the first surface to the second surface.

7. The absorbent article of claim 6, wherein the pore size increases by at least about 25 percent from the first surface to the second surface of the porous separation layer.

8. The absorbent article of claim 6, wherein the pore size increase by about 30 to about 1000 percent from the first surface of the porous separation layer to the second surface of the porous separation layer.

9. The absorbent article of claim 1, wherein the pores originating on the second surface of the porous separation layer are less wettable than the pores originating on the first surface of the porous separation layer.

10. An absorbent article comprising:
a breathable liquid-impervious outer cover;
a liquid-pervious topsheet;
an absorbent core disposed between the topsheet and the breathable outer cover; and
a porous separation layer disposed between the absorbent core and the breathable outer cover,
wherein the porous separation layer includes a plurality of wettable pores originating on a first surface adjacent the absorbent core and a plurality of less wettable pores originating on a second surface adjacent the breathable outer cover, the pores originating on the second surface having a larger average pore size proximate the second surface than the pores originating on the first surface.

11. The absorbent article of claim 10, wherein the pores originating on the first surface of the porous separation layer are at least about 5 percent more wettable than the pores originating on the second surface of the porous separation layer.

12. The absorbent article of claim 10, wherein the pores originating on the first surface of the porous separation layer are about 10 to about 95 percent more wettable than the pores originating on the second surface of the porous separation layer.

13. The absorbent article of claim 10, wherein the pores originating on the first surface of the porous separation layer and the pores originating on the second surface of the porous separation layer define a decreasing wettability gradient from the first surface to the second surface.

14. The absorbent article of claim 13, wherein the wettability decreases by at least about 5 percent from the first surface of the porous separation layer to the second surface of the porous separation layer.

15. The absorbent article of claim 13, wherein the wettability decreases by about 10 to about 95 percent from the first surface of the porous separation layer to the second surface of the porous separation layer.

16. The absorbent article of claim 10, wherein the pores originating on the second surface of the porous separation layer have an average pore size proximate the second surface about 30 to about 1000 percent greater than pores originating on the first surface of the porous separation layer.

17. The absorbent article of claim 10, wherein the pores originating on the first surface of the porous separation layer have an average pores size proximate the first surface of at least about 100 microns.

18. The absorbent article of claim 10, wherein the pores originating on the second surface of the porous separation layer have an average pore size proximate the second surface up to about 1000 microns.

19. The absorbent article of claim 10, wherein the pores originating on the first surface of the porous separation layer and the pores originating on the second surface of the porous separation layer define an increasing pore size gradient from the first surface to the second surface.

20. The absorbent article of claim 19, wherein the pore size increase by about 30 to about 1000 percent from the first surface of the porous separation layer to the second surface of the porous separation layer.

21. An absorbent article comprising:
a breathable liquid-impervious outer cover;
a liquid-pervious topsheet;
an absorbent core disposed between the topsheet and the breathable outer cover; and
a porous separation layer disposed between the absorbent core and the breathable outer cover,
wherein the porous separation layer includes a plurality of wettable pores originating on a first surface adjacent the absorbent core and a plurality of less wettable pores originating on a second surface adjacent the breathable outer cover defining a decreasing wettability gradient from the first surface and the second surface, the pores originating on the second surface having a larger average pore size proximate the second surface than the pores originating on the first surface.

22. The absorbent article of claim 21, wherein the wettability decreases by about 10 to about 95 percent from the first surface of the porous separation layer to the second surface of the porous separation layer.

23. The absorbent article of claim 21, wherein the pores originating on the second surface of the porous separation layer have an average pore size about 30 to about 1000 percent greater than the pores originating on the first surface of the porous separation layer.

24. The absorbent article of claim 21, wherein the pores originating on the first surface of the porous separation layer have an average pore size proximate the first surface of at least about 100 microns.

25. The absorbent article of claim 21, wherein the pores originating on the second surface of the porous separation layer have an average pore size proximate the second surface up to about 1000 microns.

26. The absorbent article of claim 21, wherein the pores originating on the first surface of the porous separation layer and the pores originating on the second surface of the porous separation layer define an increasing pore size gradient from the first surface to the second surface.

27. An absorbent article comprising:
a liquid-impervious breathable outer cover;
a liquid-pervious topsheet;
an absorbent core including a plurality of apertures disposed between the topsheet and the breathable outer cover; and
a porous separation layer positioned between the absorbent core and the breathable outer cover,
wherein the porous separation layer includes a plurality of wettable pores having an average pore size of at least about 100 microns originating on a first surface adjacent the absorbent core and a plurality of larger, less wettable pores originating on a second surface adjacent the breathable outer cover, the pores originating on the first surface and the pores originating on the second surface defining a wettability gradient from the first surface to the second surface and a pore size gradient from the first surface to the second surface.

28 The absorbent article of claim 27 wherein at least a portion of the apertures of the absorbent core correspond to at least a portion of the pores originating on the first surface of the porous separation layer.

29. The absorbent article of claim 27 wherein the wettability decreases by about 10 to about 95 percent from the first surface of the porous separation layer to the second surface of the porous separation layer.

30. The absorbent article of claim 27, wherein the pore size increases by about 30 to about 1000 percent from the first surface of the porous separation layer to the second surface of the porous separation layer.
